(19) United States
(12) Plant Patent Application Publication
(10) Pub. No.: US 2010/0122387 P1
(43) Pub. Date: May 13, 2010

(54) Strawberry plant named 'Reliance'

(50) ;

(75) Inventors: Stephen M. Ackerman, Salinas, CA (US); Steven D. Nelson, Watsonville, CA (US); Michael D. Nelson, Watsonville, CA (US)

Correspondence Address: FOLEY AND LARDNER LLP;SUITE 500, 3000 K STREET NW, WASHINGTON, DC, 20007, US

(73) Assignees: PLANT SCIENCES, INC.; BERRY R&D, INC.

(21) Appl. No.: 12/455,165

(22) Filed: May 29, 2009

Related U.S. Application Data
(60) Provisional application No. 61/114,194, filed on Nov. 13, 2008.

(51) Int. Cl. A01H 5/00

(52) U.S. Cl. PLT/208

(57) ABSTRACT

This invention relates to a new and distinct variety of strawberry plant named ‘RELIANCE’. This new strawberry plant named ‘RELIANCE’ is primarily adapted to the growing conditions of the central coast of California, and is primarily characterized by its large to very large fruit size; red to dark red fruit color; high productivity; medium sized plant; calyx that tends to be more equal to the diameter of the fruit; uniformly shaped, conical berries with good flavor; and relatively short fruiting trusses

### LATIN NAME OF THE GENUS AND SPECIES OF THE PLANT CLAIMED

Fragaria ananassa

### VARIETY DENOMINATION

‘RELIANCE’

### BACKGROUND OF THE INVENTION

The present invention relates to a new and distinct strawberry variety named ‘RELIANCE’. This new variety is a result of a controlled cross made in 2000 in an ongoing breeding program between strawberry variety designated ‘PS-592’ (patented, U.S. Plant Pat. No. 9,903) and strawberry variety designated ‘PS-1269’ (patented, U.S. Plant Pat. No. 10,686) in an ongoing breeding program. Due to the combining of the reciprocal seed lots, it is unknown as to which parent variety is the seed parent and which parent variety is the pollen parent. The variety is botanically known as Fragaria ananassa.

The seedling resulting from the aforementioned cross was selected from a controlled breeding plot in Monterey County, Calif. in the summer of 2002. After its selection, the new variety was asexually propagated by stolons in both San Joaquin County, Calif., and Siskiyou County, Calif. The new variety was extensively tested over the next several years in fruiting fields in Monterey County, Calif. This propagation has demonstrated that the combination of traits disclosed herein as characterizing the new variety are fixed and remain true to type through successive generations of asexual reproduction.

### BRIEF SUMMARY OF THE INVENTION

‘RELIANCE’ is primarily adapted to the climate and growing conditions of the central coast of California. The nearby Pacific Ocean provides the needed humidity and moderate temperatures to produce a strong vigorous plant and maintain fruit quality during the spring, summer and fall production months.

The following traits have been repeatedly observed and are determined to be unique characteristics of ‘RELIANCE’, which in combination distinguish this strawberry plant as a new and distinct variety:

1. Large to very large fruit size;

2. Red to dark red fruit color;

3. High productivity;

4. Medium sized plant;

5. Calyx that tends to be more equal to the diameter of the fruit;

6. Uniformly shaped, conical berries with good flavor; and

7. Relatively short fruiting trusses

The strawberry varieties that are believed to be most closely related to the new strawberry variety ‘RELIANCE’ are the strawberry variety ‘PS-4634’ (patented, U.S. Plant Pat. No. 17,487) and the strawberry variety ‘PS-5298’ (patented, U.S. Plant Pat. No. 19,583). In side-by-side comparisons to the similar strawberry varieties ‘PS-4634’ and ‘PS-5298’, the new strawberry variety ‘RELIANCE’ differs by the following combination of characteristics as described in Table 1:

TABLE 1

| | | ‘PS-4634’ | ‘PS-5298’ |
|---|---|---|---|
| Characteristic | ‘RELIANCE’ | (US PP 17,487) | (US PP 19,583) |
| 1. Color of mature fruit | Ranges from | Ranges from | Red |
| | red to dark red | red to orange-red | |
| 2. Average fruit weight (gm) | 27.4 | 27.6 | 24.6 |
| 3. Marketable yield (gm/plt) | 1,646 | 1,521 | 1,445 |
| 4. Size of calyx in relation | Ranges from same size | Same size | Slightly larger |
| to fruit diameter | to slightly smaller | | |
| 5. Plant size | Medium | Large | Large |
| 6. Stolon anthocyanin intensity | Weak | Ranges from weak | Weak |
| | | to medium | |
| 7. Interveinal leaf blistering | Medium | Ranges from | Ranges from |
| | | medium to strong | medium to strong |
| 8. Foliage gloss | Medium | Ranges from | Ranges from |
| | | medium to strong | medium to strong |
| 9. Petiole length (cm) | 19.4 | 23.7 | 24.4 |
| 10. Petiolule length (mm) | 15.5 | 9.4 | 12.3 |
| 11. Terminal leaf shape of base | Obtuse | Acute | Acute |
| 12. Ternunal leaf shape of teeth | Rounded | Obtuse | Obtuse |
| 13. Fruiting truss length | 29.9 | 39.0 | 36.3 |
| at maturity (cm) | | | |
| 14. Fruiting truss pubescence | Strong | Ranges from | Medium |
| | | medium to weak | |

For identification a series of AFLP molecular markers have been determined for this new variety.

‘RELIANCE’ differs from its parents, ‘PS-592’ and ‘PS-1269’ by the following combination of characteristics as described in Table 2.

TABLE 2

| | | ‘PS-592’ | ‘PS-1269-2 |
|---|---|---|---|
| Characteristic | ‘RELIANCE’ | (US PP 9,903) | (US PP 10,686) |
| 1. Color of mature fruit | Ranges from | Ranges from | Red |
| | red to dark red | red to orange-red | |
| 2. Fruit size | Ranges from | Medium | Medium |
| | large to very large | | |
| 3. Plant size | Medium | Large | Large |
| 4. Fruit skin firmness | Ranges from | Medium | Medium |
| | medium to strong | | |
| 5. Fruit gloss | Medium | Ranges from | Ranges from |
| | | medium to strong | medium to weak |
| 6. Fruit productivity | High | Ranges from | Medium |
| | | medium to high | |

### BRIEF DESCRIPTIONS OF THE PHOTOGRAPHS

The accompanying color photographs illustrate the overall appearance of typical specimens of the new strawberry variety ‘RELIANCE’, at various stages of development as true as it is reasonably possible with color reproductions of this type. Color in the photographs may differ slightly from the color value cited in the botanical description which accurately describe the color of ‘RELIANCE’. The depicted plant and plant parts of the new strawberry variety ‘RELIANCE’ were taken in Monterey County, Calif., and are approximately 8 to 9 months old:

FIG. 1 shows typical fruiting field characteristics of ‘RELIANCE’ taken in the month of June 2008;

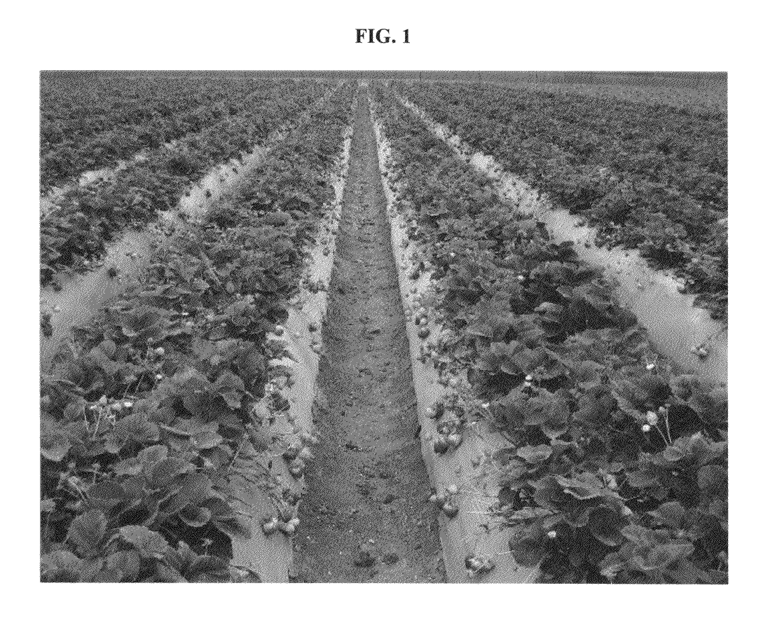

FIG. 2 shows a close-up view of a typical leaf structure of ‘RELIANCE’ taken in the month of July 2008;

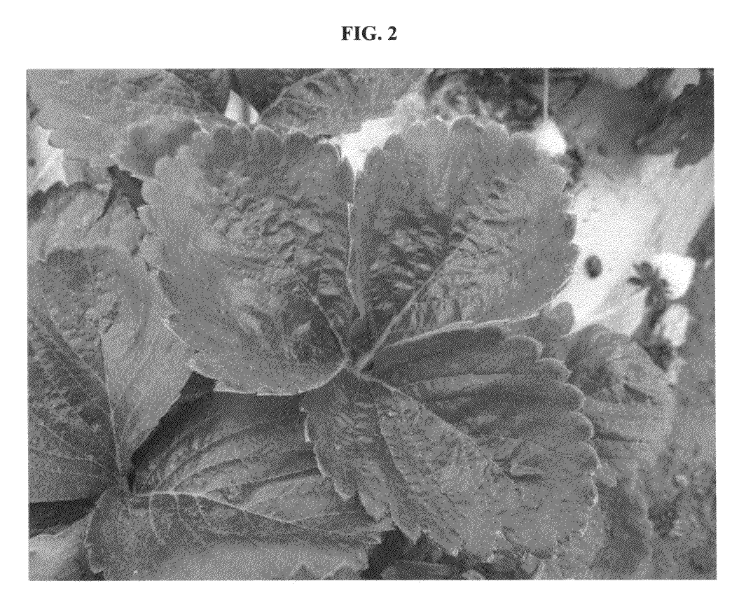

FIG. 3 shows typical mature and immature field fruit of ‘RELIANCE’ taken in the month of July 2008;

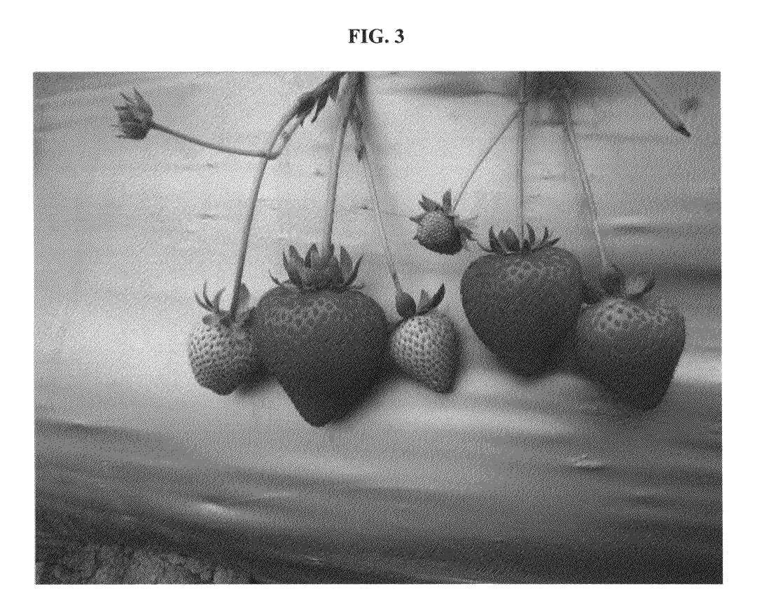

FIG. 4 shows a close-up view of mature fruit of ‘RELIANCE’ taken in the month of July 2008;

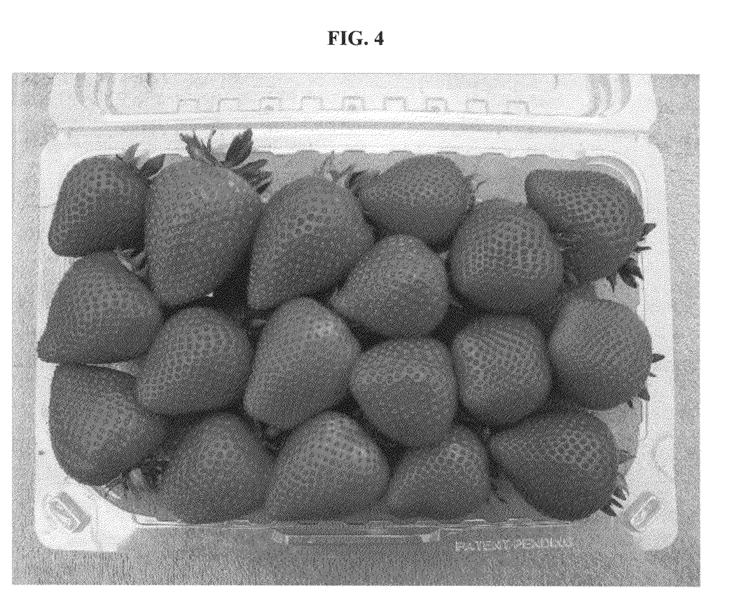

FIG. 5 shows typical internal and external mature fruit characteristics of ‘RELIANCE’ taken in the month of July 2008.

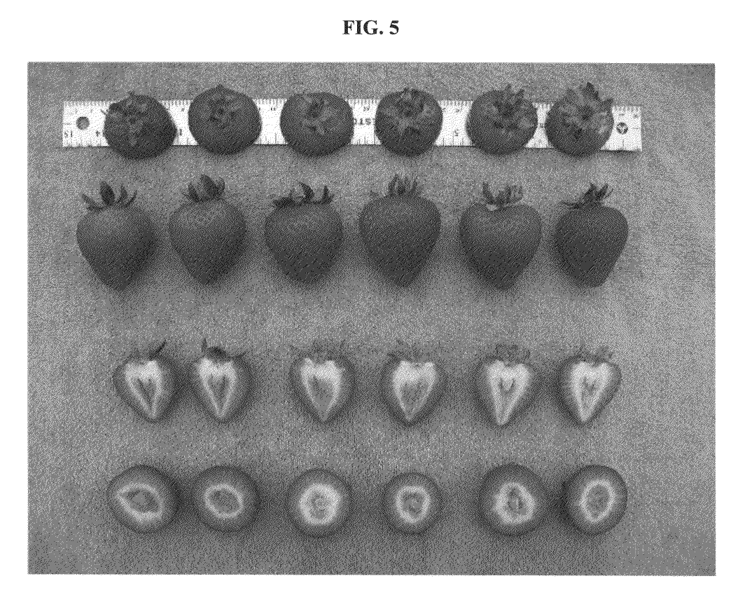

### DETAILED BOTANICAL DESCRIPTION

The new variety ‘RELIANCE’ has not been observed under all possible environmental conditions. The characteristics of the new variety ‘RELIANCE’ may vary in detail, depending upon variations in environmental factors, including weather (temperature, humidity and light intensity), day length, soil type and location. In addition, the characteristics of any parental variety or comparison variety included in Tables 1-9 of the present invention, may vary in detail, depending upon variations in environmental factors, including weather (temperature, humidity and light intensity), day length, soil type and location.

The aforementioned photographs, together with the following description of the new variety ‘RELIANCE’, unless otherwise noted, is based on observations taken during the 2008 growing season in Monterey County, Calif. These measurements and ratings were taken from plants of ‘RELIANCE’ dug from a high-elevation nursery located in Siskiyou County, Calif. during the middle of October 2007 and planted approximately 3 weeks later in Monterey County, Calif. The approximate age of the observed plants is 8 to 10 months. Yield observations and fruit quality characteristics are averaged from four years of data collected from the 2005 through 2008 growing seasons. Flower measurements and characteristics are from secondary flowers unless otherwise noted. Fruit characteristics and measurements are from secondary fruit unless otherwise noted.

Color terminology where noted follows The Royal Horticultural Society Colour Chart, London (2007).

The following Tables 3 to 9 describe fruit, plant, stolon, foliage, fruiting truss, flower and pest disease characteristics of the new strawberry ‘RELIANCE’ in comparison to the similar strawberry variety ‘PS-4634’ (patented, U.S. Plant Pat. No. 17,487) and strawberry variety ‘PS-5298’ (patented, U.S. Plant Pat. No 19,583).

TABLE 3
FRUIT CHARACTERISTICS

| | | ‘PS-4634’ | ‘PS-5298’ |
|---|---|---|---|
| Characteristic | ‘RELIANCE’ | (US PP 17,487) | (US PP 19,583) |
| Color of mature fruit | RHS 46A | RHS 45B | RHS 45A |
| | Ranges from red to | Red to orange-red | Red |
| | dark red | | |
| Color of internal flesh | RHS 44A | RHS 44C | RHS 44B |
| | Medium red | Ranges from medium | Medium red |
| | | to light red | |
| Fruit length (cm) | 4.7 | 4.9 | 4.6 |
| Fruit width (cm) | 4.2 | 4.1 | 4.0 |
| Fruit length/width ratio | 1.12 | 1.19 | 1.15 |
| | Slightly longer | Slightly longer | Slightly longer |
| | than broad | than broad | than broad |
| Calyx diameter (cm) | 3.9 | 4.4 | 4.2 |
| Average fruit weight (gm) | 27.4 | 27.6 | 24.6 |
| Achene color, shaded side | RHS 162B | RHS 162C | RHS 160B |
| | Greyed-yellow group | Greyed-yellow group | Greyed-yellow group |
| Achene color, sun exposed side | RHS 184B | RHS 184C | RHS 184C |
| | Greyed-purple group | Greyed-purple group | Greyed-purple group |
| Achenes per berry | 340 | 359 | 268 |
| Achene weight (mg) | 0.60 | 0.45 | 0.50 |
| Marketable fruit yield (gm/plt) | 1,646 | 1,521 | 1,445 |
| Fruit Size | Ranges from large | Ranges from large | Ranges from medium |
| | to very large | to very large | to large |
| Predominant fruit shape | Conical | Conical | Conical |
| Difference in shapes | Slight | Moderate | Moderate |
| between primary and | | | |
| secondary fruit | | | |
| Band without achenes | Narrow | Narrow | Ranges from |
| | | | absent to very narrow |
| Unevenness of fruit surface | Weak | Ranges from weak | Weak |
| | | to medium | |
| Evenness of fruit color | Ranges from slightly | Ranges from slightly | Ranges from slightly |
| | uneven to even | uneven to even | uneven to even |
| Fruit glossiness | Medium | Strong | Ranges from |
| | | | medium to strong |
| Insertion of achenes | Level with surface | Level with surface | Level with surface |
| Insertion of calyx | Ranges from level | Ranges from level | Ranges from level |
| | with to in the basin | with to in the basin | with to above fruit |
| Attitude of the calyx | Ranges from spreading | Ranges from spreading | Ranges from spreading |
| | to reflexed | to reflexed | to reflexed |
| Size of calyx in relation | Ranges from same size | Same size | Slightly larger |
| to fruit diameter | to slightly smaller | | |
| Adherence of calyx | Strong | Strong | Strong |
| Firmness of fruit skin | Ranges from | Strong | Ranges from |
| | medium to strong | | medium to strong |
| Firmness of fruit flesh | Ranges from | Medium | Ranges from |
| | medium to firm | | medium to firm |
| Keeping quality | Good | Very good | Good |
| Distribution of red color | Marginal and central | Marginal and central | Marginal and central |
| of the flesh | | | |
| Hollow center expression | Moderate | Strong | Moderate |
| Fruit flavor | Good | Very good | Good |
| Soluble solids (% brix) | 8.8 | 9.6 | 9.6 |
| Time of first flowering | Medium | Early | Early |
| Flowering period | Early to mid March | Late February to early | Late February to early |
| | to December | March to December | March to December |
| Time of first harvesting | Medium | Early | Early |
| Harvest period | Early to mid April | Late March to early | Late March to early |
| | to December | April to December | April to December |
| Type of bearing | Partially everbearing | Not remontant | Partially everbearing |

TABLE 4
PLANT CHARACTERISTICS

| | | ‘P5-4634’ | ‘PS-5298’ |
|---|---|---|---|
| Characteristic | ‘RELIANCE’ | (US PP 17,487) | (US PP 19,583) |
| Plant height (cm) | 26.5 | 31.9 | 34.1 |
| Plant spread (cm) | 33.8 | 36.7 | 40.0 |
| Crowns/plant | 5.3 | 6.0 | 6.7 |
| Plant size | Medium | Large | Large |
| Plant habit | Globose | Globose | Globose |
| Plant density | Medium | Medium | Medium |
| Plant vigor | Medium | Strong | Strong |

TABLE 5
STOLON CHARACTERISTICS

| | | ‘PS-4634’ | ‘PS-5298’ |
|---|---|---|---|
| Characteristic | ‘RELIANCE’ | (US PP 17,487) | (US PP 19,583) |
| Stolon color | RHS 146C | RHS 144A | RHS 145A |
| | Yellow-green group | Yellow-green group | Yellow-green group |
| Stolon anthocyanin coloration | RHS 180C | RHS 182D | RHS 182D |
| | Greyed-red group | Greyed-red group | Greyed-red group |
| Stolon anthocyanin intensity | Weak | Ranges from weak | Weak |
| | | to medium | |
| Average stolon number | 1.1 | 2.7 | 0.9 |
| per plant from the | | | |
| fruiting field | | | |
| Stolon diameter at bract (mm) | 4.3 | 4.5 | 4.1 |
| Stolon pubescence | Ranges from | Ranges from | Ranges from |
| | medium to strong | medium to weak | medium to strong |
| Attitude of hairs | Slightly outward | Upward | Slightly outward |

TABLE 6
FOLIAGE CHARACTERISTICS

| | | ‘PS-4634’ | ‘PS-5298’ |
|---|---|---|---|
| Characteristic | ‘RELIANCE’ | (US PP 17,487) | (US PP 19,583) |
| Foliage: | | | |
| Color of upper surface | RHS 137B | RHS 137A | RHS 137B |
| | Medium green | Medium green | Medium green |
| Color of under side | RHS 147C | RHS 147C | RIIS 147C |
| | Yellow-green group | Yellow-green group | Yellow-green group |
| Shape in cross section | Ranges from strongly | Ranges from slightly | Ranges from slightly |
| | concave to flat | concave to flat | concave to flat |
| Interveinal blistering | Medium | Ranges from | Ranges from |
| | | medium to strong | medium to strong |
| Leaf glossiness | Medium | Ranges from | Ranges from |
| | | medium to strong | medium to strong |
| Number of leaflets | Three | Three | Three |
| Terminal Leaflet: | | | |
| Length (cm) | 9.5 | 10.5 | 10.2 |
| Width (cm) | 9.1 | 8.0 | 8.8 |
| Lengtblwidth ratio | 1.04 | 1.32 | 1.16 |
| | Ranges from as long | Much longer | Much longer |
| | as broad to longer | than broad | than broad |
| | than broad | | |
| Serrations/leaf | 22.1 | 21.9 | 19.1 |
| Leaf size | Ranges from | Ranges from | Ranges from |
| | medium to large | medium to large | medium to large |
| Shape of base | Obtuse | Acute | Acute |
| Shape of teeth | Rounded | Obtuse | Obtuse |
| Petiole: | | | |
| Petiole Color | RHS 145A | RHS 145A | RHS 144A |
| | Yellow-green group | Yellow-green group | Yellow-green group |
| Petiole length (cm) | 19.4 | 23.7 | 24.4 |
| Petiole diameter (mm) | 5.0 | 5.4 | 5.4 |
| Petiolule color | RHS 145A | RHS 145A | RHS 144A |
| | Yellow green group | Yellow green group | Yellow green group |
| Petiolule length (mm) | 15.5 | 9.4 | 12.3 |
| Petiole pubescence | Moderate | Ranges from | Ranges from |
| | | moderate to sparce | moderate to sparce |
| Attitude of hairs | Ranges from upward to | Slightly outward | Strongly outward |
| | slightly outward | | |
| Stipule: | | | |
| Color | RHS 146B | RHS 146B | RHS 146B |
| | Yellow-green group | Yellow-green group | Yellow-green group |
| Anthocyanin | RHS 61B | RHS 58A | RHS 61B |
| coloration | Red-purple group | Red-purple group | Red-purple group |
| Anthocyanm intensity | Medium | Strong | Weak |
| Length (mm) | 19.4 | 19.5 | 23.5 |
| Width (mm) | 8.3 | 9.5 | 9.8 |

TABLE 7
FRUITING TRUSS CHARACTERISTICS

| | | ‘P5-4634’ | ‘PS-5298’ |
|---|---|---|---|
| Characteristic | ‘RELIANCE’ | (US PP 17,487) | (US PP 19,583) |
| Anthocyanin coloration | RHS 181B | RHS 182D | RHS 182C |
| | Greyed-red group | Greyed-red group | Greyed-red group |
| Anthocyanin intensity | Ranges from | Absent or very weak | Ranges from |
| | medium to weak | | medium to weak |
| Length at maturity (cm) | 29.9 | 39.0 | 36.3 |
| Position relative to foliage | Beneath the foliage | Level with | Level with to |
| | | | beneath the foliage |
| Pubescence | Strong | Ranges from | Medium |
| | | medium to weak | |
| Attitude at first pick | Prostrate | Prostrate | Prostrate |

TABLE 8
FLOWER CHARACTERISTICS

| | | ‘PS-4634’ | ‘PS-5298’ |
|---|---|---|---|
| Characteristic | ‘RELIANCE’ | (US PP 17,487) | (US PP 19,583) |
| Petal color | RHS 155C | RHS 155C | RHS 155C |
| | White | White | White |
| Sepal color | RHS 137B | RHS 137A | RHS 137B |
| | Green group | Green group | Green group |
| Receptacle color | RHS 147C | RHS 147C | RHS 147C |
| | Yellow-green group | Yellow-green group | Yellow-green group |
| Corolla diameter (mm) | 30.6 | 34.2 | 31.8 |
| Calyx diameter (mm) | 32.0 | 36.6 | 36.5 |
| Petallength(mm) | 12.3 | 12.8 | 12.0 |
| Petal width (mm) | 12.8 | 12.5 | 12.5 |
| Petal length/width ratio | 0.96 | 1.03 | 0.95 |
| Petals/flower | 5.9 | 6.0 | 6.4 |
| Sepal length (mm) | 12.1 | 13.2 | 13.1 |
| Sepal width (mm) | 4.8 | 5.2 | 4.5 |
| Sepal length/width ratio | 2.53 | 2.53 | 2.92 |
| Sepals/flower | 11.6 | 12.3 | 13.0 |
| Size of calyx relative to | Ranges from | Larger | Larger |
| corolla | same size to larger | | |
| Size of inner calyx | Ranges from | Ranges from | Ranges from |
| relative to outer calyx | same size to smaller | same size to larger | same size to smaller |
| Relative position of petals | Overlapping | Overlapping | Overlapping |

TABLE 9
PEST AND DISEASE REACTIONS

| | | ‘PS-4634’ | ‘PS-5298’ |
|---|---|---|---|
| Characteristic | ‘RELIANCE’ | (US PP 17,487) | (US PP 19,583) |
| Two spotted spider | Moderately | Moderately | Moderately |
| mite | susceptible | susceptible | susceptible |
| Flower thrips | Moderately | Moderately | Moderately |
| | resistant | susceptible | susceptible |
| Powdery mildew | Moderately | Moderately | Moderately |
| | susceptible | resistant | susceptible |
| Botrytis fruit rot | Moderately | Moderately | Moderately |
| | susceptible | susceptible | susceptible |
| Angular leaf spot | Susceptible | Susceptible | Moderately |
| | | | susceptible |

We claim:

1. A new and distinct strawberry plant named ‘RELIANCE’, as herein described and illustrated by the characteristics set forth above.
